(19) United States
(12) Patent Application Publication
(10) Pub. No.: US 2012/0171300 A1
(43) Pub. Date: Jul. 5, 2012

(54) Durable Antimicrobial Composition Including a Surfactant

(76) Inventors: David William Koenig, Menasha, WI (US); Jeremy David Paulsen, Neenah, WI (US); Corey Thomas Cunningham, Larsen, WI (US); Rebecca Ann Vongsa, Neenah, WI (US)

(21) Appl. No.: 13/269,922

(22) Filed: Oct. 10, 2011

Related U.S. Application Data
(63) Continuation-in-part of application No. 12/982,058, filed on Dec. 30, 2010.

(51) Int. Cl. A61K 33/40; A61P 31/10; A61P 31/12; A61P 31/04

(52) U.S. Cl. 424/616

(57) ABSTRACT

The present invention relates to compositions having durable antimicrobial activity. The compositions include a carbonate/bicarbonate salt of a quaternary ammonium cation, an organic acid, hydrogen peroxide, a surfactant and a polymer. The polymer is selected from cationic amine polymer-epichlorohydrin adduct, cationic amine polymer-epichlorohydrin resin, poly(methacrylamidopropyltrimethylammonium)chloride, poly(bis(2-chloroethyl)ether-alt-1,3-bis(dimethylamino)propyl)urea, poly(diallyldimethylammonium)chloride, poly(t-butyl acrylate co-ethyl acrylate co-methacrylic acid), polyethylene oxide, polyquaternium-16, polyquaternium-22, polyquatemium-67 and combinations thereof. The surfactant is selected from cationic surfactants, non-ionic surfactants, zwitterionic surfactants and combinations thereof.

### RELATED APPLICATION DATA

This application is a continuation-in-part application claiming priority from presently copending U.S. application Ser. No. 12/982,058 entitled “DURABLE ANTIMICROBIAL COMPOSITION” filed on Dec. 30, 2010, in the names of Corey T. Cunningham et al.

### FIELD

Compositions having durable antibacterial activity are disclosed herein. The compositions include a carbonate/bicarbonate salt of a quaternary ammonium cation, an organic acid, hydrogen peroxide, a polymer, and a surfactant. The polymer is selected from cationic amine polymer-epichlorohydrin adduct, cationic amine polymer-epichlorohydrin resin, poly(methacrylamidopropyltrimethylammonium)chloride, poly(bis(2-chloroethyl)ether-alt-1,3-bis(dimethylamino)propyl)urea, poly(diallyldimethylammonium)chloride, poly(t-butyl acrylate co-ethyl acrylate co-methacrylic acid), polyethylene oxide, polyquaternium-16, polyquaternium-22, polyquatemium-67, and mixtures of these polymers. The surfactant is selected from cationic surfactants, non-ionic surfactants, zwitterionic surfactants, and combinations thereof.

### BACKGROUND

In order to protect health and maintain hygiene, a variety of environments require controlled and limited microbial growth. Such environments include temporary and permanent healthcare facilities, caregiver facilities (e.g., daycares, nursing homes, etc.) and households. When growth of potentially harmful microbes is not controlled/limited in these environments, the risk of infection and spread of disease increases. Infection and disease may compromise the health and safety of humans and/or animals occupying these environments. While potentially not as sensitive as the above-identified environments, workplace and public environments may also be negatively impacted by uncontrolled/unlimited growth of disease-causing microbes.

Some types of microorganisms (bacteria, viruses, fungi, etc.) are capable of negatively impacting the health and/or safety of living organisms. Such microorganisms can be transmitted by contact with surfaces on which the microorganisms are present and/or multiplying and by contact between humans/animals already infected with particular microorganisms. When such microorganisms spread and infect new “hosts”, the “host” can either go from an otherwise healthy state to a state of illness or from a “compromised” state (i.e., a state of pre-existing illness or a weak immune system) to a more serious/severe state. The public health impact of the undesired spread of microorganisms is significant as reflected by time out of school, time away from work (either for self or to care for others not able to care for themselves), additional time for which professional health care is needed, etc. Therefore, it is desirable to be able to prevent or inhibit microbial presence/growth on targeted surfaces. The presence of microorganisms can be eliminated/controlled using surface treatments that may be applied directly (as from a spray bottle) and by using wipes or other carriers that include the surface treatment. Further, it is desirable that such surface treatments have durability and persistence so that they do not need to be re-applied on a frequent basis.

There are many detergent, disinfectant, cleaning and antimicrobial compositions known in the art for killing and preventing growth of microorganisms. These compositions include components/ingredients that are well-known for antimicrobial functionality. For example, quaternary ammonium compounds are considered “broad spectrum” antimicrobial cationic compounds that are effective against both Gram positive (e.g., Staphylococcus species) and Gram negative (e.g., Escherichia coli) microorganisms. Other components/ingredients that may be incorporated into products for removing/reducing microorganisms on surfaces include alcohols, acids and bleaching agents, such as hydrogen peroxide. Not all of the antimicrobial components can be used at the same time because some of them form unstable combinations.

Disinfecting and cleaning compositions that provide antimicrobial activity over a period of time are also known in the art. For example, U.S. Pat. No. 6,270,754 issued to Zhou et al. and entitled “Antimicrobial Cleaning Composition” (hereinafter “the '754 patent”) is directed to an antibacterial cleaning composition that exhibits germicidal activity for sustained periods of time. The '754 patent discloses an aqueous cleaning composition that includes a quaternary ammonium compound, an anionic polymer (where the anionic polymer has an acid number greater than 10 and the anionic polymer is partially or completely neutralized by the quaternary ammonium compound to form a polymer complex), a dispersing agent and/or a water-miscible solvent. The aqueous cleaning composition of the '754 patent has antibacterial activity against both Gram positive and Gram negative bacteria. However, the components of the '754 patent may not be effective against a broader range of microorganisms, such as non-enveloped viruses.

In addition to the composition of the '754 patent, there are compositions known in the art that are effective against a broad spectrum of microorganisms and continue to have activity for a period of time. For example, U.S. Pat. No. 7,598,214 issued to Cusack et al. and entitled “Disinfecting Compositions Containing A Polymer Complex Of an Organic Acid” (hereinafter “the '214 patent”) is directed to compositions that include at least one organic acid and at least one polymer capable of forming a complex with the at least one organic acid. The compositions of the '214 patent may also optionally include an anionic surfactant and an organic acid. The organic acid may be citric acid and examples of suitable polymers include vinylpyrrolidone/dimethylaminoethylmethacrylate copolymer, vinylpyrrolidone/vinylacetate copolymers, vinylpyrrolidone/vinylcaprolactum/ammonium derivative terpolymers and polyvinylpyrrolidone. The compositions of the '214 patent need an organic acid and they are pH sensitive. Because of the acid-based reaction between the polymer having a tertiary amine functionality and the organic acid, the compositions are not effective in higher pH environments. In a higher pH environment, the reaction would reverse and the polymer would be rendered ineffective because it would be neutral. Though the compositions of the '214 patent are effective against a broader spectrum of microorganisms, the compositions may not be effective against the spore-form of all microorganisms because the compositions cannot penetrate through the outer wall of the spores.

While many antimicrobial compositions are known and while some of those compositions maintain their antimicrobial activity over a period of time, there remains a need in the art for a durable antimicrobial composition that is effective against a broad range of microorganisms, including the spore-form of potentially harmful microorganisms. Additionally, there remains a need for a durable antimicrobial composition that is stable (i.e., is not reactive) so that it is not unnecessarily harsh (causing wear or corrosion) on the surfaces on which it is used. Further, there remains a need for a durable antimicrobial composition that does not require a volatile solvent that may have an unpleasant smell.

Furthermore, there is a need to provide a durable antimicrobial composition that includes a stable surfactant such that cleaning is achieved and also provides a composition wherein a long-lasting biocide effect is maintained, rapid broad spectrum germicidal properties are maintained, and multiple fragrances can be employed for consumer acceptance.

### SUMMARY

Durable antimicrobial compositions that are effective against a broad range of potentially harmful microorganisms and that do not have to be reapplied on a frequent basis to the surfaces on which controlled microbial growth is desired are described herein. The durable antimicrobial compositions are effective against a broad range of microorganisms, including the spore-form of microorganisms, because of the compositions components, which are unexpectedly stable in combination with each other. Additionally, the durable antimicrobial compositions do not need to contain a volatile solvent that could make the compositions unpleasant to use. Additionally, the compositions need to include a surfactant to provide a cleaning benefit without losing efficacy or the long-lasting benefit of the compositions.

In one aspect, the compositions have durable antimicrobial activity and include a carbonate/bicarbonate salt of a quaternary ammonium cation, an organic acid, hydrogen peroxide, a surfactant and a polymer. The polymer is selected from cationic amine polymer-epichlorohydrin adduct, cationic amine polymer-epichlorohydrin resin, poly(methacrylamidopropyltrimethylammonium) chloride, poly(bis(2-chloroethyl)ether-alt-1,3-bis(dimethylamino)propyl)urea, poly(diallyldimethylammonium)chloride, poly(t-butyl acrylate co-ethyl acrylate co-methacrylic acid), polyethylene oxide, polyquaternium-16, polyquaternium-22, polyquaternium-67, and mixtures of such polymers. As described herein, the compositions have a durable or persistent activity to kill and prevent the growth of potentially harmful microorganisms. The durability of the compositions is indicated by the compositions retaining antimicrobial activity after twenty-five insults of E. coli organisms as measured by a log 2 reduction in organisms upon the twenty-fifth insult of 10⁶ total organisms. The durable antimicrobial compositions are stable; the stability of the compositions is reflected by the compositions maintaining their efficacy during shelf-life studies. For example, the compositions remain effective (meaning, they have the same level of durability to effect a log 2 reduction in organisms after twenty-five insults of 10⁶ organisms) after storage for three months at 40° C.; further, the compositions remain effective after storage for one month at 50° C., nine months at 25° C. and after three freeze-thaw cycles. The compositions are liquid at room temperature and can be applied directly to a surface for which it is desired to prevent or inhibit microbial growth. The compositions may be applied using a spray bottle or other known structure for dispensing liquids. Alternatively, the compositions may be applied to a surface by transfer from a basesheet, such as a wiper, into which a representative composition has been incorporated. The basesheet may be made of a nonwoven material or of a cellulosic material. More particularly, the composition may include from 0.2 to 15.0 percent by weight of the carbonate/bicarbonate salt of a quaternary ammonium cation. The composition may include from 0.1 to 3.0 percent by weight of the organic acid, which may be selected from citric, malic, maleic, oxalic, glutaric, succinic, lactic, glycolic, fumaric, acetic, benzoic, propionic, sorbic, tartaric, formic, and mixtures of such organic acids. The composition may include from 0.5 to 5.0 percent by weight of hydrogen peroxide and the composition may include from 0.5 to 10.0 percent by weight of polymer.

These aspects and additional aspects of the invention will be described in greater detail herein. Further, it is to be understood that both the foregoing general description and the following detailed description are exemplary and are intended to provide further explanation of the invention claimed.

### DETAILED DESCRIPTION

The present disclosure of the invention will be expressed in terms of its various components, elements, constructions, configurations, arrangements and other features that may also be individually or collectively referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

Compositions having durable antimicrobial activity are disclosed herein. The compositions may be used to kill or to inhibit the growth of microorganisms that are potentially harmful or capable of causing disease. The durable antimicrobial compositions do not need to contain a volatile solvent and therefore, do not generate an unpleasant smell when used. The compositions are effective at killing and/or inhibiting growth of a broad range of microorganisms. For example, the compositions are effective against both Gram positive and Gram negative bacteria. Additionally, the compositions are effective against viruses, fungi, mildew and mold. Further, the compositions are effective against bacteria that form spores, bacteria with waxy outer layers, fungi that form spores (fungal spores) and enveloped and non-enveloped viruses. Without wishing to be bound by theory, it is believed that the compositions are capable of breaking down the waxy outer layer of a bacteria or outer layer of a spore so that the compositions can penetrate into the microorganism beyond the outer layer.

The durable antimicrobial compositions may be used to control microbial growth on a variety of surfaces, including relatively durable objects having both hard and soft surfaces; for example, appropriate surfaces may include door knobs, light switches, countertops, sinks, wash basins, telephones, keyboards, remote controls, medical instruments, upholstery, curtains, bedspreads, towels and shoes. The compositions may be applied to the targeted surface either directly, in liquid form, such as by a spray bottle or similar packaging capable of delivering a liquid composition in a relatively uniform amount over the full surface to be covered. Alternatively, the composition may be applied to the targeted surface by a carrier, such as a basesheet (i.e., a “wet” wipe or wiper). Because the compositions are liquid at room temperature, the composition may be applied to a surface by wiping the surface with a basesheet that has been saturated with the composition; the composition will transfer from the basesheet to the surface. The basesheet may be formed from one or more woven materials, nonwoven materials, cellulosic materials, and combinations of such materials. More specifically, the basesheet may be formed of nonwoven fibrous sheet materials that include meltblown, spunlace, coform, air-laid, bonded-carded web materials, hydroentangled materials, and combinations of such materials. Such materials can be made of synthetic or natural fibers or a combination of such fibers. Typically, the basesheet will have a basis weight of from 25 to 120 grams per square meter and desirably from 40 to 90 grams per square meter.

The basesheet may be constructed of a coform material of polymer fibers and absorbent fibers having a basis weight of from 45 to 80 grams per square meter and desirably 60 grams per square meter. Typically, such coform basesheets are constructed of a gas-formed matrix of thermoplastic polymeric meltblown fibers and cellulosic fibers. Various suitable materials may be used to provide the polymeric meltblown fibers, such as, for example, polypropylene microfibers. Alternatively, the polymeric meltblown fibers may be elastomeric polymer fibers, such as those provided by a polymer resin. For instance, VISTAMAXX elastic olefin copolymer resin designated PLTD-1810, available from ExxonMobil Corporation of Houston, Tex., or KRATON G-2755, available from Kraton Polymers of Houston, Tex., may be used to provide stretchable polymeric meltblown fibers for the coform basesheets. Other suitable polymeric materials or combinations thereof may alternatively be utilized as known in the art.

The coform basesheet additionally may be constructed of various absorbent cellulosic fibers, such as, for example, wood pulp fibers. Suitable commercially available cellulosic fibers for use in the coform basesheets can include, for example, NF 405, which is a chemically treated bleached southern softwood Kraft pulp, available from Weyerhaeuser Co. of Federal Way, Wash.; NB 416, which is a bleached southern softwood Kraft pulp, available from Weyerhaeuser Co.; CR-0056, which is a fully debonded softwood pulp, available from Bowater, Inc. of Greenville, S.C.; Golden Isles 4822 debonded softwood pulp, available from Koch Cellulose of Brunswick, Ga.; and SULPHATATE HJ, which is a chemically modified hardwood pulp, available from Rayonier, Inc. of Jessup, Ga. The relative percentages of the polymeric meltblown fibers and cellulosic fibers in the coform basesheet may vary over a wide range depending upon the desired characteristics of the wipes. For example, the coform basesheet may have from 10 to 90 weight percent, desirably from 20 to 60 weight percent, and more desirably from 25 to 35 weight percent of polymeric meltblown fibers based on the dry weight of the coform basesheet.

The durable antimicrobial compositions may be incorporated into the basesheet in an add-on amount of from 50 to 800 percent by weight of the basesheet. More specifically, the compositions may be incorporated into the basesheet in an add-on amount of from 200 to 600 percent by weight of the basesheet or from 400 to 600 percent by weight of the basesheet. The composition add-on amounts may vary depending on the composition of the basesheet.

The present invention relates to compositions having durable antimicrobial activity. The “durability” or “persistence” of antimicrobial activity is descriptive of a benefit provided by the durable antimicrobial compositions. From a cost and efficiency standpoint, it is desirable to maintain antimicrobial activity on a surface over a period of time with one application of a composition rather than having to frequently apply a composition because its antimicrobial activity rapidly dissipates. From a public health standpoint, a durable antimicrobial composition is desirable because such a composition is more likely to prevent microbial growth than a composition that is weaker to begin with and a durable antimicrobial composition introduces less liquid/material into the environment, thereby decreasing the opportunity for microbes to develop resistance. The durability of the compositions is measured by activity after twenty-five (25) insults with a representative Gram negative bacterium, Escherichia coli (E. coli). The compositions retain activity sufficient to cause a log 2 reduction upon the twenty-fifth insult of 10⁶ total E. coli organisms. Additionally, the durability of the compositions is measured by ability to effect greater log 2 reduction against Gram positive bacteria, Gram negative bacteria, enveloped viruses, non-enveloped viruses, fungi, mildew and mold twenty-four (24) hours after application of the composition to a surface. Further, the durability of the durable antimicrobial compositions is measured by ability to effect greater log 2 reduction in microorganisms in the presence of soil after either of the first two assays described above (i.e., (1) twenty-fifth insult of 10⁶ total organisms; or (2) twenty-four hours after application). From a practical standpoint, a standard surface, such as a countertop, table, telephone, etc., in a susceptible environment, such as a hospital or daycare facility, is continuously exposed to potentially harmful microorganisms. Given the rate at which exposure to new microorganisms typically occurs, a durable antimicrobial composition may be applied to the surface in a timeframe of every 24 hours to 48 hours in order to kill and/or to prevent the growth of microorganisms. Comparatively, an antimicrobial composition that is not durable would need to be applied continuously to a surface to maintain a comparable level of antimicrobial activity. In a less susceptible environment and with a less susceptible surface, such as draperies in a home, the durable antimicrobial composition may last up to seven days at full activity.

The durable antimicrobial compositions include a carbonate/bicarbonate salt of a quaternary ammonium cation. Quaternary ammonium compounds are generally considered “broad spectrum” antimicrobial cationic compounds that have efficacy against both Gram positive and Gram negative microorganisms. The carbonate/bicarbonate salts of quaternary ammonium cations may be selected from dioctyldimethylammonium carbonate, decyloctyldimethylammonium carbonate, didecyldimethylammonium carbonate, benzalkonium carbonate, benzethonium carbonate, stearalkonium carbonate, cetrimonium carbonate, behentrimonium carbonate, dioctyldimethylammonium bicarbonate, decyloctyldimethylammonium bicarbonate, didecyldimethylammonium bicarbonate, benzalkonium bicarbonate, benzethonium bicarbonate, stearalkonium bicarbonate, cetrimonium bicarbonate, behentrimonium bicarbonate, and mixtures of one or more such carbonate salts. The durable antimicrobial compositions may include from 0.2 to 15.0 percent by weight of one or more carbonate/bicarbonate salts of quaternary ammonium cations.

The durable antimicrobial compositions also include an organic acid. Organic acids are also known to have efficacy against the growth of microorganisms. The organic acid may be selected from citric, malic, maleic, oxalic, glutaric, succinic, lactic, glycolic, fumaric, acetic, benzoic, propionic, sorbic, tartaric, formic and mixtures of one or more such organic acids. The durable antimicrobial compositions may include from 0.1 percent by weight to 3.0 percent by weight of one or more organic acids.

Additionally, the durable antimicrobial compositions include hydrogen peroxide. The hydrogen peroxide is stable in the durable antimicrobial compositions, despite the presence of the carbonate/bicarbonate salt. Existing antimicrobial compositions do not contain stabilized hydrogen peroxide in combination with a carbonate/bicarbonate salt. The stability of the hydrogen peroxide is measured by the durable antimicrobial compositions maintaining their initial concentration and efficacy during shelf-life studies. For example, the compositions remain effective (meaning, they have the same level of durability to effect a log 2 reduction in organisms after twenty-five insults of 10⁶ organisms) after storage for three months at 40° C.; further, the compositions remain effective after storage for one month at 50° C., nine months at 25° C. and after three freeze-thaw cycles. Specifically, the concentration of hydrogen peroxide in exemplary compositions after various shelf-life studies is provided in Table 1 below. The exemplary compositions in Table 1 each included the following components: (1) 2 percent by weight of CARBOQUAT H solution as available from Lonza Group Limited Switzerland; (2) 0.85 percent by weight of citric acid;

(3) 3 percent by weight hydrogen peroxide; and (4) 2.5 percent by weight polyquaternium-22 polymer (MERQUAT 295 polymer available from Nalco Company). The compositions in Table 1 also included 0.4 percent by weight urea; further, the compositions included the indicated type and amount of organic solvent and the remainder of the compositions was water. Note, while an organic solvent was used for purposes of these examples, the solvent is not needed for the compositions to have the described efficacy and durability. In fact, before the treated surfaces were insulted as described below, the treated surfaces were allowed to dry and the solvent and water would have evaporated. Each of these compositions produced the indicated log reduction of microorganisms within five minutes after twenty-five individual insults of 10⁶ E. coli organisms.

TABLE 1

| | | Wt. % of | Wt. % of H₂O₂ | Wt. % of | Wt. % of | Wt. % of |
|---|---|---|---|---|---|---|
| | | H₂O₂ at | after 3 | H₂O₂ after | H₂O₂ after | H₂O₂ after |
| | Wt. % and type of | Time | Freeze/Thaw | 1 Week at | 1 Month at | 3 Months at |
| Example | Solvent | Zero | Cycles | 50° C. | 40° C. | 25° C. |
| 1 | 0% Ethanol | 3.15 | 2.94 | 2.92 | 3.07 | Not tested |
| 2 | 2.5% Ethanol | 3.12 | 3.14 | 2.94 | 3.07 | Not tested |
| 3 | 5.0% Ethanol | 3.13 | 3.14 | 2.93 | 3.08 | Not tested |
| 4 | 7.5% Ethanol | 3.10 | 3.10 | 2.92 | 3.04 | Not tested |
| 5 | 5.0% Ethylene Glycol | 3.11 | 3.09 | 2.86 | 3.02 | Not tested |
| 6 | 5.0% Propylene Glycol | 3.10 | 3.10 | 2.85 | 3.02 | 3.10 |
| 7 | 5.0% Butylene Glycol | 3.06 | 3.04 | 2.82 | 2.98 | Not tested |
| 8 | 5.0% Butyl Cellosolve | 3.11 | 3.09 | 2.88 | 3.03 | Not tested |

The results in Table 1 show that durable antimicrobial compositions described herein are stable as indicated by sustained presence of hydrogen peroxide under different shelf-life study conditions.

While not wishing to be bound by theory, it is believed that the ability to provide compositions with stabilized hydrogen peroxide significantly expands the range of microorganisms that the durable antimicrobial compositions are effective against. Some microorganisms exist or are spread in spore form, where the spores have an outer layer; the outer layer presents a barrier to penetration by some conventional antimicrobial compositions. It is believed that the stabilized hydrogen peroxide in the durable antimicrobial compositions is capable of penetrating the outer layer of spores, thereby facilitating exposure of the spore interior to the carbonate/bicarbonate salt of a quaternary ammonium cation. The carbonate/bicarbonate salt of a quaternary ammonium cation prevents future germination or development of the spore. The durable antimicrobial compositions may include from 0.5 to 5.0 percent by weight of hydrogen peroxide.

The stability of the durable antimicrobial compositions is also measurable by the ongoing/sustained detectable concentration of the carbonate/bicarbonate salt of a quaternary ammonium cation, organic acid and hydrogen peroxide components of the compositions. The carbonate/bicarbonate salt of a quaternary ammonium cation component may be detected using high pressure liquid chromatography (HPLC) with an evaporative light-scattering (ELS) detector. The mobile phase for the HPLC is an acidic mixture of acetonitrile and water. The organic acid component may be detected using HPLC with an ultra-violet (UV) absorption detector monitoring the 220 nanometer wavelength. The mobile phase for the HPLC for the detection of the organic acid is also an acidic mixture of acetonitrile and water. The hydrogen peroxide component may be detected by titrating the sample with a solution of ceric sulfate and ferroin indicator as described in the journal article, Frank P. Greenspan and Donald G. MacKellar entitled “Analysis of Aliphatic Per Acids” published in Analytical Chemistry, 1948, 20, 1061. The durable antimicrobial compositions have a sustained and detectable presence of these components after experiencing the accelerated shelf-life conditions described herein.

The durability of the hydrogen peroxide in the presence of the carbonate/bicarbonate salt is provided by the polymer component of the durable antimicrobial composition. The polymer is selected from cationic amine polymer-epichlorohydrin adduct, cationic amine polymer-epichlorohydrin resin, poly(methacrylamidopropyltrimethylammonium)chloride, poly(bis(2-chloroethyl)ether-alt-1,3-bis(dimethylamino)propyl)urea, poly(diallyldimethylammonium)chloride, poly(t-butyl acrylate co-ethyl acrylate co-methacrylic acid), polyethylene oxide, polyquaternium-16, polyquaternium-22, polyquaternium-67 and mixtures of such polymers. The durable antimicrobial compositions may include from 0.5 to 10 percent by weight of polymer.

Additionally, the durable antimicrobial compositions also include a compatible surfactant. The surfactant is selected from cationic surfactants, non-ionic surfactants, zwitterionic surfactants, and combinations thereof. The durable antimicrobial composition may suitably include one or more compatible surfactants in an amount of from about 0.01 to about 10 percent by weight of the composition.

Compatible surfactants provide adequate stability with regard to freeze/thaw, temperature extremes, and fragrance addition. Furthermore, these compatible surfactants allow for basal biocide sequestration after drying followed by a metered burst release performance upon wetting. Not all surfactants are compatible with durable antimicrobial composition. For example, an anionic surfactant will react with other components of the durable antimicrobial composition to form a coacervate. This leads to poor resistance to abrasion and poor efficacy.

As described above, the surfactant may be a nonionic surfactant. Nonionic surfactants typically have a hydrophobic base, such as a long chain alkyl group or an alkylated aryl group, and a hydrophilic chain comprising a certain number (e.g., 1 to about 30) of ethoxy and/or propoxy moieties. Examples of some classes of nonionic surfactants that can be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈₋₁₈) acids, condensation products of ethylene oxide with long chain amines or amides, condensation products of ethylene oxide with alcohols, and combinations thereof.

Various specific examples of suitable nonionic surfactants for use in the durable antimicrobial composition include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, ethoxylated fatty (C₁₋₂₂) alcohol, including 3 to 20 ethylene oxide moieties, polyoxyethylene-20 isohexadecyl ether, polyoxyethylene-23 glycerol laurate, PEG-80 sorbitan laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG-12 dioleate, PEG-8 dioleate, and combinations thereof.

Additional nonionic surfactants that can be used include water soluble alcohol ethylene oxide condensates, such as the condensation products of a secondary aliphatic alcohol containing between about 8 to about 18 carbon atoms in a straight or branched chain configuration condensed with between about 5 to about 30 moles of ethylene oxide. Such nonionic surfactants are commercially available under the trade name Tergitol from The Dow Chemical Company (Midland, Mich.). Specific examples of such commercially available nonionic surfactants of the foregoing type are C₁₁₋₁₅ secondary alkanols condensed with either 9 moles of ethylene oxide (Tergitol 15-S-9) or 12 moles of ethylene oxide (Tergitol 15-S-12) marketed by The Dow Chemical Company (Midland, Mich.).

Other suitable nonionic surfactants include the polyethylene oxide condensates of one mole of alkyl phenol containing from about 8 to 18 carbon atoms in a straight or branched chain alkyl group with about 5 to 30 moles of ethylene oxide. Specific examples of alkyl phenol ethoxylates include nonyl condensed with about 9.5 moles of ethylene oxide per mole of nonyl phenol, dinonyl phenol condensed with about 12 moles of ethylene oxide per mole of phenol, dinonyl phenol condensed with about 15 moles of ethylene oxide per mole of phenol and diisoctylphenol condensed with about 15 moles of ethylene oxide per mole of phenol. Commercially available nonionic surfactants of this type include Igepal CO-630 (a nonyl phenol ethoxylate) marketed by ISP Corp. (Wayne, N.J.). Suitable non-ionic ethoxylated octyl and nonyl phenols include those having from about 7 to about 13 ethoxy units. Such compounds are commercially available under the trade name Triton X by The Dow Chemical Company (Midland, Mich.).

Alkyl polyglycosides may also be used as a nonionic surfactant in the durable antimicrobial composition. Suitable alkyl polyglycosides are known nonionic surfactants that are alkaline and electrolyte stable. Alkyl mono and polyglycosides are prepared generally by reacting a monosaccharide, or a compound hydrolyzable to a monosaccharide with an alcohol such as a fatty alcohol in an acid medium. Commercially available nonionic surfactants of this type include Glucopon 425 marketed BASF (Ludwidschafen, Germany).

Suitable zwitterionic surfactants for use in the durable antimicrobial composition include, for example, alkyl amine oxides, silicone amine oxides, and combinations thereof. Various specific zwitterionic surfactants for use in the durable antimicrobial composition include, for example, Almondamidopropylamine Oxide, Babassuamidopropylamine Oxide, Behenamine Oxide, Cocamidopropylamine Oxide, Cocamine Oxide, Dihydroxyethyl Cocamine Oxide, Dihydroxyethyl Lauramine Oxide, Dihydroxyethyl Stearamine Oxide, Isostearamidopropylamine Oxide, Isostearamidopropyl Morpholine Oxide, Lauramidopropylamine Oxide, Decylamine Oxide, Lauramine Oxide, Methyl Morpholine Oxide, Myristamidopropylamine Oxide, Myristamine Oxide, Palmitamidopropylamine Oxide, Palmitamine Oxide, PEG-3 Lauramine Oxide, Soyamidopropylamine Oxide, Stearamidopropylamine Oxide, Stearamine Oxide, and combinations thereof. Commercially available alkyl amine oxide surfactants of this type include Mackamine CO (Cocamine Oxide) marketed by McIntyre Group, A Rhodia Company, (University Park, Ill.). Suitable cationic surfactants for use in the durable antimicrobial composition include, for example, alkyl ammonium salts, polymeric ammonium salts, alkyl pyridinium salts, aryl ammonium salts, alkyl aryl ammonium salts, silicone quaternary ammonium compounds, and combinations thereof. Specific examples of cationic surfactants include behenyltrimonium chloride, stearlkonium chloride, distearalkonium chloride, chlorohexidine diglutamate, polyhexamethylene biguanide (PHMB), cetyl pyridinium chloride, benzammonium chloride, benzalkoniumchloride, and combinations thereof.

In addition to the components described herein, the durable antimicrobial compositions may also include a polar carrier solvent, pH adjuster, fragrance, preservative, dye, corrosion inhibitor, builder, cleansing solvent, and other components known to be useful in antimicrobial compositions. The durable antimicrobial compositions may include from 67 to 98 percent by weight of one or more of these other components.

While other blending methods may be used, an example of one method of blending the durable antimicrobial compositions is as follows: (1) Add water to vessel for mixing of the components of the compositions; (2) Slowly add the carbonate/bicarbonate salt of the quaternary ammonium cation component to the vessel; (3) Slowly add the organic acid component to the vessel and begin mixing at low revolutions per minute (RPM) (i.e., 150-250 RPM); (4) Continue mixing until any foam that is present dissipates (e.g., up to 10 minutes for a 1 liter batch); (5) If desired for additional stability when the final composition is applied to a surface, add a stabilizer such as urea and continue mixing at low RPM (e.g., add 0.4 percent by weight of urea if adding 3.0 percent by weight hydrogen peroxide); (6) Slowly add hydrogen peroxide to the vessel and continue mixing at low RPM; (7) Slowly add the polymer component to the vessel and continue mixing at low RPM; (8) If desired for solution clarity, an appropriate organic solvent (e.g., ethanol, isopropanol, ethylene glycol, propylene glycol, butylene glycol, ethylene glycol monobutyl ether, etc.) may slowly be added to the vessel; and (9) If necessary, adjust the pH of the final composition in the vessel to pH 3.0 (+/−0.25) with a dilute (10 to 25 percent by weight) solution of potassium hydroxide. Those of skill in the art will appreciate that there are other methods by which the components of the durable antimicrobial compositions may be blended. However, it is an aspect of the present invention that the carbonate/bicarbonate salt of the quaternary ammonium cation is neutralized by the addition of the organic acid in step 3 prior to the addition of the hydrogen peroxide.

Representative examples of the polymers of the durable antimicrobial composition are provided in Table 2 below. Each exemplary polymer described in Table 2 was used in a durable antimicrobial composition that included the following components: (1) 2 percent by weight of CARBOQUAT H solution as available from Lonza Group Limited Switzerland; (2) 0.85 percent by weight of citric acid; and (3) 3 percent by weight hydrogen peroxide. The compositions also included 0.4 percent by weight urea and 20 percent ethanol; the remainder was water. Note, while ethanol was used for purposes of these examples, the ethanol is not needed for the durable antimicrobial compositions to have the described efficacy and durability. In fact, before the treated surfaces were insulted as described below, the treated surfaces were allowed to dry and the ethanol and water would have evaporated. Similarly, while not required, the urea is added to provide enhanced stability of the compositions after application to a surface. Each of these compositions produced the indicated log reduction of microorganisms within five minutes after twenty-five and/or fifty individual insults of 10⁶ E. coli organisms.

TABLE 2

| | | | | | Log | Log |
|---|---|---|---|---|---|---|
| | | Trade Name or | | | Reduction | Reduction |
| | | Molecular Weight | | Wt. % | after 25 | after 50 |
| Example | Polymer Name | Range | Supplier | polymer | insults | insults |
| 1 | cationic amine polymer- | Crepetrol 970 | Ashland | 5.0 | 5.6 | 4.7 |
| | epichlorohydrin adduct | | | | | |
| 2 | cationic amine polymer- | Crepetrol X-cell | Ashland | 5.0 | 6.1 | 4.7 |
| | epichlorohydrin resin | | | | | |
| 3 | poly(methacrylamidopropyl | MAQUAT PQ-125 | Mason | 2.5 | 6.8 | 6.0 |
| | trimethylammonium) | | Chemical | | | |
| | Chloride | | Company | | | |
| 4 | poly(bis(2-chloroethyl)ether- | Polyquaternium-2, | Rhodia | 5.0 | 2.3 | no data |
| | alt 1,3,bis (dimethylamino) | Mirapol A-15 | | | | |
| | propyl) urea | | | | | |
| 5 | poly(bis(2-chloroethyl)ether- | Polyquaternium-2, | Rhodia | 2.5 | 6.6 | 6.0 |
| | alt 1,3,bis (dimethylamino) | Mirapol A-15 | | | | |
| | propyl) urea | | | | | |
| 6 | poly(diallyldimethyl | MW 200000-350000 | Sigma- | 5.0 | 5.7 | 4.7 |
| | ammonium) chloride | | Aldrich | | | |
| 7 | poly(diallyldimethyl | MW 400000-500000 | Sigma- | 2.5 | no data | 6.3 |
| | ammonium) chloride | | Aldrich | | | |
| 8 | poly(diallyldimethyl | MW 400000-500000 | Sigma- | 7.5 | no data | 6.0 |
| | ammonium) chloride | | Aldrich | | | |
| 9 | poly(t-butyl acrylate co-ethyl | MW ~100000 | Sigma- | 5.0 | 2.5 | no data |
| | acrylate co-methacrylic acid) | | Aldrich | | | |
| 10 | polyethylene oxide | MW ~300000 | Sigma- | 2.5 | 2.4 | no data |
| | | | Aldrich | | | |
| 11 | Polyquaternium-16 | Luviquat | BASF | 5.0 | 4.7 | 2.8 |
| | | Excellence | | | | |
| 12 | Polyquaternium-16 | Luviquat Style | BASF | 7.5 | no data | 6.1 |
| 13 | Polyquaternium-16 | Luviquat | BASF | 2.5 | 2.9 | no data |
| | | Excellence | | | | |
| 14 | Polyquaternium-22 | Merquat 295 | Nalco | 2.0 | 6.0 | 4.7 |
| | | | Company | | | |
| 15 | Polyquaternium-22 | Merquat 295 | Nalco | 1.0 | no data | 6.3 |
| | | | Company | | | |
| 16 | Polyquaternium-22 | Merquat 295 | Nalco | 3.0 | no data | 6.3 |
| | | | Company | | | |
| 17 | Polyquaternium-67 | SoftCAT Polymer | Dow | 5.0 | 2.0 | no data |
| | | SX-400 | Chemical | | | |

The “no data” designations indicate insult conditions that were not tested either because (i) the polymer at a lower wt. % already demonstrated at least a log 3 reduction after twenty-five insults or (ii) the polymer demonstrated close to a log 2 reduction after twenty-five insults and therefore, was unlikely to demonstrate a log 2 reduction after fifty insults.

In addition to the log reduction data for the durable antimicrobial compositions provided in Table 2 above, stability data for some of the compositions is provided in Table 3 below. Stability is demonstrated by a continued presence of hydrogen peroxide in the compositions after exposure to different shelf-life conditions. Each of the compositions in Table 3 contained components similar to those described for the compositions of Table 2.

TABLE 3

| | | | Wt. % of | Wt. % of | Wt. % of | Wt. % of |
|---|---|---|---|---|---|---|
| | | Wt. % of | H₂O₂ after | H₂O₂ after | H₂O₂ after | H₂O₂ after |
| | | H₂O₂ at | 2 Weeks at | 4 Weeks at | 1 Month at | 3 Months |
| Example | Wt. % and type of Polymer | Time Zero | 50° C. | 50° C. | 40° C. | at 40° C. |
| 1 | 2.5% Poly(methyacryl- | 2.99 | 2.99 | 2.88 | 3.03 | 2.90 |
| | amidopyltriethyl ammonium) | | | | | |
| | Chloride | | | | | |
| 2 | 1.0% Polyquaternium-22 | 3.05 | 2.98 | 2.90 | 3.04 | 2.89 |
| 3 | 7.5% Polyquaternium-16 | 2.97 | 2.90 | 2.74 | 2.95 | 2.58 |

The results in Table 3 show that the durable antimicrobial compositions are stable as indicated by sustained presence of hydrogen peroxide under different shelf-life study conditions.

Samples were also prepared to illustrate the stability of the composition with a surfactant included. To show stability, tests were run to determine the release profile of different biocides contained in exemplary durable antimicrobial formulations on hard surfaces and the durability of the films obtained from such formulations. To illustrate the release profile of the various compositions, sample compositions were prepared as described below. To test durability, pre-weighed substrates were placed in a fume-hood and 80-microliters of durable antimicrobial formulation was dispensed on top of each substrate spreading the formulation across the entire surface. The substrate is a non-porous, pre-cleaned, passivated 1 inch by 1 inch piece of stainless steel (18 ga 304 sst with mirrored finish). Substrates were passivated by immersing substrates in the Carboquat-H 3 wt. % solution for 2 hours; immersing them in the citric acid 3 wt. % solution for 2 hours and rinsing substrates twice by immersing them in clean DI-water for 30 minutes; and immersing them in the 3 percent hydrogen peroxide solution for 2 hours and rinsing the substrates twice immersing by them in clean DI-water for 30 minutes. After application of the formulation, the substrates were dried for 14 hours and then weighed to determine the weight of the film.

The substrates were extracted with 1.2 mL of DI water in a weighing dish placed on orbital-shaker (IKA Shuttler MTS4). The substrate is placed into the DI water with the polymer film facing the weighing dish, in the water and the speed of the orbital shaker is set to 100. Aliquots of 300 microliters each of the extraction fluid are then transferred to HPLC vials at 15 seconds, 30 seconds, 1 minute, 2 minutes and 7.5 minutes. An HPLC system with UV- and ELSD-detectors; and a Neptune Hilic Silica Column (5μ 100 A; 15 cm by 4.6 mm) from ES-Industries (Cat. #135221-NPN-SI) was used to determine the amount of the biocide released from the system at each time. The UV-Detector was set at 195 nm and the ELSD-Detector was set at a gas flow of 0.6, with a Neb. Temperature of 100° C. and an Evap. Temperature of 80° C. The gradient was set at 0.08 percent TFA with an injection volume of 10 μL.

Representative examples of a surfactant of the durable antimicrobial composition are provided in Table 4 below. Each exemplary surfactant described in Table 4 was used in a durable antimicrobial composition in a concentration of 1.0 percent active surfactant by weight that included the following components: (1) 2 percent by weight of CARBOQUAT H solution as available from Lonza Group Limited Switzerland; (2) 0.85 percent by weight of citric acid; (3) 3 percent by weight hydrogen peroxide; and (4) 2.5 percent by weight polyquaternium-22 polymer (MERQUAT 295 polymer available from Nalco Company). The compositions also included 0.4 percent by weight urea and 20 percent ethanol; the remainder was water. Table 4 also illustrates the biocide release of various actives of the durable antimicrobial compositions.

TABLE 4

| | | | % Release of citric | % Release of Carboquat |
|---|---|---|---|---|
| Example | Surfactant | Trade Name | acid after 2 minutes | H after 2 minutes |
| 1 | None | N/A | 76% | 82% |
| 2 | coco-glucoside | Glucopon 425N | 67% | 74% |
| 3 | decylamine oxide | Mackamine C-10 | 91% | 82% |
| 4 | alcohol ethoxylate | Ecosurf EH-6 | 91% | 68% |
| 5 | C₁₁₋₁₅ pareth-9 | Tergitol 15-S-9 | 83% | 63% |

The durable antimicrobial compositions including the exemplary surfactants represented in Table 4 have a similar release profile of biocide and similar durability described for the exemplary durable antimicrobial composition not including a surfactant in Table 4. All of the durable antimicrobial compositions including the exemplary surfactants had quick release of the citric acid and CARBOQUAT H compound illustrating the compositions are stable and similar durability required of the durable antimicrobial composition described herein.

While the compositions of the invention have been described in detail with respect to specific aspects thereof, it will be appreciated that those skilled in the art, upon attaining an understanding of the foregoing, may readily conceive of alterations to, variations of and equivalents to these compositions. Accordingly, the scope of the present invention should be assessed as that of the claims and any equivalents thereto.

We claim:

1. A composition having durable antimicrobial activity comprising:
a carbonate/bicarbonate salt of a quaternary ammonium cation;
an organic acid;
a surfactant selected from cationic surfactants, non-ionic surfactants, zwitterionic surfactants, and combinations thereof;
hydrogen peroxide; and
a polymer selected from cationic amine polymer-epichlorohydrin adduct, cationic amine polymer-epichlorohydrin resin, poly(methacrylamidopropyltrimethylammonium)chloride, poly(bis(2-chloroethyl)ether-alt-1,3-bis(dimethylamino)propyl)urea, poly(diallyldimethylammonium)chloride, poly(t-butyl acrylate co-ethyl acrylate co-methacrylic acid), polyethylene oxide, polyquatemium-16, polyquaternium-22, polyquaternium-67, and combinations thereof.

2. The composition of claim 1 wherein the carbonate/bicarbonate salt of a quaternary ammonium cation is selected from dioctyldimethylammonium carbonate, decyloctyldimethylammonium carbonate, didecyldimethylammonium carbonate, benzalkonium carbonate, benzethonium carbonate, stearalkonium carbonate, cetrimonium carbonate, behentrimonium carbonate, dioctyldimethylammonium bicarbonate, decyloctyldimethylammonium bicarbonate, didecyldimethylammonium bicarbonate, benzalkonium bicarbonate, benzethonium bicarbonate, stearalkonium bicarbonate, cetrimonium bicarbonate, behentrimonium bicarbonate, and combinations thereof.

3. The composition of claim 1 wherein the organic acid is selected from citric, malic, maleic, oxalic, glutaric, succinic, lactic, glycolic, fumaric, acetic, benzoic, propionic, sorbic, tartaric, formic, and combinations thereof.

4. The composition of claim 1 wherein the composition further comprises urea.

5. The composition of claim 1 wherein the composition retains antimicrobial activity after twenty-five insults of E. coli as measured by a log 2 reduction in organisms upon the twenty-fifth insult of 10⁶ total organisms.

6. The composition of claim 1 wherein the composition is effective against Gram positive bacteria, Gram negative bacteria, enveloped viruses, non-enveloped viruses, fungi, fungal spores, mildew and mold.

7. The composition of claim 1 wherein the composition includes from 0.2 to 15.0 percent by weight of the carbonate/bicarbonate salt of a quaternary ammonium cation.

8. The composition of claim 1 wherein the composition includes from 0.1 to 3.0 percent by weight of the organic acid.

9. The composition of claim 1 wherein the composition includes from 0.5 to 5.0 percent by weight of hydrogen peroxide.

10. The composition of claim 1 wherein the composition includes from 0.5 to 10.0 percent by weight of polymer.

11. The composition of claim 1 wherein the composition is incorporated into a nonwoven basesheet.

12. The composition of claim 1 wherein the surfactant is selected from ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈₋₁₈) acids, condensation products of ethylene oxide with long chain amines or amides, condensation products of ethylene oxide with alcohols, alkyl ammonium salts, polymeric ammonium salts, alkyl pyridinium salts, aryl ammonium salts, alkyl aryl ammonium salts, silicone quaternary ammonium compounds, alkyl amine oxides, silicone amine oxides, and combinations thereof.

13. The composition of claim 1 wherein the composition includes from 0.05 to 3 percent by weight of the surfactant.
